(19) United States
(12) Patent Application Publication
(10) Pub. No.: US 2014/0072642 A1
(43) Pub. Date: Mar. 13, 2014

(54) ENDOTHELIAL PROGENITOR CELLS LABELLED WITH SUPERPARAMAGNETIC IRON OXIDE MICRO AND NANOPARTICLES IN CEREBROVASCULAR DISORDERS, PROCESS FOR THEIR PREPARATION AND USES THEREOF

(71) Applicants: Joan Montaner Villalonga, Barcelona (ES); Anna Rosell Novel, Barcelona (ES); Anna Roig Serra, Bellaterra (ES)

(72) Inventors: Joan Montaner Villalonga, Barcelona (ES); Anna Rosell Novel, Barcelona (ES); Anna Roig Serra, Bellaterra (ES)

(73) Assignees: CONSEJO SUPERIOR DE INVESTIGACIONES CIENTIFICAS, Madrid (ES); FUNDACIO HOSPITAL UNIVERSITARI VALL D'HEBRON INSTITUT DE RECERCA, Barcelona (ES)

(21) Appl. No.: 14/110,532

(22) PCT Filed: Mar. 12, 2012
(86) PCT No.: PCT/EP12/54198

§ 371 Date: Nov. 26, 2013

(30) Foreign Application Priority Data
Apr. 8, 2011 (EP) 11382104.5

(51) Int. Cl. A61K 41/00

(52) U.S. Cl. CPC A61K 41/0052
USPC 424/490; 424/93.7; 424/646; 435/325

(57) ABSTRACT

The invention relates to the use of endothelial progenitor cells (EPC) that comprises superparamagnetic particle aggregates which are able for the preparation of a medicament. Particular nanoparticle aggregates with a coating of compounds with net negative charge, and the method for obtaining them are also disclosed.

The present invention relates to the field of therapeutics, namely to the treatment of cerebrovascular ischemia and stroke, and to the products employed for minimizing the severity of the injury with the aim of restoring or resembling the normal activity of the brain.

### BACKGROUND ART

A stroke, also called a cerebrovascular accident (CVA) is the rapidly developing loss of brain function due to unbalance in the blood supply to the brain. This lack of normal blood flow can be caused by the obstruction of an artery by an embolism or blood clot or by the sudden rupture of an artery causing an haemorrhage. The affected area of the brain cannot normally function, leading to certain inabilities, such as the movement of one or more limbs on one side of the body (hemiplegia), the inability to listen or to understand or formulate speech (aphasia), or the inability to normally see.

Endothelial progenitor cells (EPC) are a type of cell population with the ability to circulate, proliferate and mature to become endothelial cells (EC) under certain conditions. Unmature EPCs do not display EC markers nor have the ability to form endothelial-like structures with a lumen but secrete major endothelial growth factors. Mature EPCs display endothelial markers and morphology and are capable to form endothelial-like structures in vitro. It is widely known that EPCs participate in the re-endothelialization and neovascularization of injured endothelium in cases of myocardial and critical limb ischemia. However, the therapeutic potential of the EPCs appears to be limited by the number of cells able to migrate, engraft and proliferate in the injured tissue. Thus, previously to be implanted in an organism, the EPCs might be labelled with magnetic micro or nanoparticles, such as superparamagnetic iron oxide (SPIO) nanoparticles or microparticles, thus allowing their imaging and distant manipulation.

Superparamagnetism is a form of magnetism, which appears in small ferromagnetic or ferrimagnetic micro or nanoparticles, every particle consisting of one magnetic domain. A magnetic domain is a region within a magnetic material which has uniform magnetization. This means that the individual magnetic moments of the atoms are aligned with one another and point in the same direction. In micro and nanoparticles, magnetization can randomly flip (turn over) direction under the influence of temperature. The typical time between two flips is called the Néel relaxation time. In the absence of external magnetic field, when the time used to measure the magnetization of the particles is much longer than the Néel relaxation time, their magnetization appears to be in average zero: they are said to be in the superparamagnetic state. In this state, an external magnetic field is able to magnetize the particles, similarly to a paramagnet. However, their magnetic susceptibility is much larger than the one of paramagnets. Unlike ferromagnets, superparamagnets do not retain any magnetization in the absence of an externally applied magnetic field, because thermal motion causes the magnetic moment to become randomly oriented. Thus the total magnetization will drop to zero when the applied field is removed.

An interesting review regarding the potential applications of EPCs with internalized magnetic nanoparticles is depicted by Gazeau et al., “Magnetic labelling, imaging and manipulation of endothelial progenitor cells using iron oxide nanoparticles”, Future Medical Chemistry 2010, Vol. 2(3), pp. 397-408. This document discloses the use of anionic magnetic nanoparticles (AMNP) which are endocytosed by EPC. The review indicates that the nanoparticle-labelled EPCs are viable and suitable to be organized into tubular lattices, which are the precursors of vascular capillaries, and that they can be artificially obtained under the direction of external magnetic fields. Moreover, the review also shows that remotely applied magnetic forces may enable intracellular manipulation and may optimize cell-delivery strategies for localizing cell therapy to target sites. EPCs have been efficiently targeted to engineered stents, or positioned at sites of carotid arterial injury.

Nonetheless, Gazeau et al (supra), also list several of the drawbacks existing with the magnetic applications, as well as the derived needs of this field. It seems that for remote magnetic control of nanoparticle-labelled EPCs, high loads of labels of iron are required, thus compromising the cell viability or inducing toxicity. Another disadvantage derives from the cell aggregation tendency when they are carrying nanoparticles, which disturb the correct engraftment of cells in the target area.

Other experimental data performed with EPCs labelled with superparamagnetic nanoparticles are disclosed by Yang et al., “Superparamagnetic iron oxide nanoparticles may affect endothelial progenitor cell migration ability and adhesion capacity”, Cytotherapy 2010, Vol. 11, pp. 251-259. Yang et al. labelled EPCs with a type of Superparamagnetic iron oxide (SPIO) nanoparticles coated with carboxydextran and it was evaluated the intracellular iron uptake, the cell proliferation, migration, adhesion and level of apoptosis. Their results suggest that SPIO nanoparticles impair EPC migration ability and promote EPC adhesion capacity. From the teachings of Yang et al., it can be derived that it is important to investigate the biological effectivity of cells labelled with SPIO nanoparticles, because it can happen that the resulting labelled cell may not be useful for systemic cell delivery, or cannot serve to remotely apply the cells to be further delivered in a desired location.

As explained by Gazeau et al. (supra), EPC cells loaded with superparamagnetic nanoparticles may be externally conducted, namely to injured myocardial regions or surrounding arteries. Once implanted into an animal being, the cells loaded with the nanoparticles are conducted by means of an external magnetic field.

Nonetheless, a particular situation appears when the injured vascular tissue is the brain vascular system, where it is really difficult to promote the neovascularization or regeneration from a peripheral healthy area, due to the very low permeability and high selectivity of the blood brain barrier, impeding moreover the translocation of most drugs. Thus, by now an effective neuroprotector and/or neuroregenerating treatments does not exist.

One approach for conducting particles to the brain area is disclosed by Rivière et al., “Magnetic targeting of nanometric magnetic fluid liposomes (MFLs) to specific brain intravascular areas”, Radiology 2007, Vol. 244, pp. 439-448, where rhodamine-labeled magnetic fluid-loaded liposomes, are intravenously injected through a catheter inserted into the left tail vein. When a magnet is placed over a specific area of the mouse brain and the rhodamine-labeled MFLs accumulated in the mouse brain microvasculature are detected. This document demonstrates that sub-micrometric magneto-liposomes may be delivered by remote control to specific cerebrovascular areas. Nonetheless, the system disclosed by Rivière is mainly focalized to the drug-delivery with liposomes and a liposome is far related from EPCs and from the applications of this cell type, namely the obtention of mature endothelial cells, which constitute the blood vessels, or the use of these cells as MRI agents to assess cell-based therapies.

In one attempt to repair damaged neuronal tissue in a model rat, Song et al., in “Using a Neodymium Magnet to Target Delivery of Ferumoxide-labeled Human Neural Stem Cells (NSC) in a Rat Model of Focal Cerebral Ischemia”, Human Gene Theraphy—2010, Vol. 21, pp.: 603-610, disclose a novel, non-invasive method for enhancing the targeted migration of transplanted stem cells to the area of interest. The authors demonstrate that ferumoxide-labeled human NSC can migrate in an enhanced way to ischemic brain lesions, and can reduce the infarct volume by placing a magnet over the damaged brain region of rats. The rats were previously submitted to a middle cerebral artery occlusion (MCAo). The authors also show that the magnetic field does not affect the biological functions of human neural stem cells (NSC).

However, the study of Song et al. (supra) behaves some limitations. As they indicate, the histopathological findings were obtained after a short period, without functional evaluation. The immortalized line of NSC is not fully applicable in clinical therapeutic applications, since it comprises an oncogene. Moreover, it has to be kept in mind that the behaviour of an immortalized line can be quite different from a normal cell line, due to the inherent immortality features.

Document US2011070202 discloses the targeting of human neural progenitor stem cells comprising superparamagnetic iron oxide nanoparticles (Feridex®) to the site of a brain injury for use in the treatment of traumatic brain injury, Parkinson's Disease, Alzheimer's Disease and amyotrophic lateral sclerosis. In this document is mentioned that one promising form for therapy for traumatic brain injury is the use of either neural or endothelial progenitor cells. Nonetheless, US2011070202 shows application of neural stem cells for a different pathology; i.e. traumatic brain injury (TBI), which is not stroke. All these pathologies or disorders widely diverge in their causes and physiological onsets. Thus, data derivable from the application of a therapy in one of these disorders is not directly applicable to others. So that, as a way of example, the administration of infused exogenous VEGF into the lateral ventricles of mice for 7 days after TBI, significantly augmented neurogenesis and angiogenesis and reduced lesion volumes after TBI, as suggested by Thau-Zuchman et al. “Vascular endothelial growth factor increases neurogenesis after traumatic brain injury”, Journal of Cerebral Blood Flow & Metabolism—2010, Vol. 30, pp.: 1008-1016. On the contrary, animal models of cerebral ischemia (stroke) have demonstrated that early administration of VEGF (intraventricular) exacerbates ischemic damage, because of its effects on blood-brain barrier (BBB) permeability (Valable et al, “VEGF-induced BBB permeability is associated with MMP-9 activity increase in cerebral ischemia: both effects decreased by Ang-1”, Journal of Cerebral Blood Flow & Metabolism—2005, Vol. 25, pp.: 1491-1504). The same is observed in post-ischemic (1 hour) administration of intravenous recombinant human VEGF (rhVEGF165) to ischemic rats, which significantly increased BBB leakage, hemorrhagic transformation, and ischemic lesions (Zhang et al, “VEGF enhances angiogenesis and promotes blood-brain barrier leakage in the ischemic brain”, J. Clin. Invest—2000, Vol. 106, pp.: 829-838).

Nowadays, most of the nanoparticles used to be endocytosed in cells consist in a core of an iron oxide compound with superparamagnetic properties and a coating. They are mostly used as contrast agents for magnetic resonance molecular imaging. The nanoparticles most broadly referred by Gazeau et al. (supra) have a core of maghemite (γFe₂O₃) and are citrate-coated. These nanoparticles are prepared in water by alkaline co-precipitation of iron (III) and iron (II) salts to form ferric oxide, followed by a step of adsorption of citrate anions to the ferric oxide surface. This method gives raise to a population of nanoparticles or nanoparticle aggregates, wherein the aggregate size mean is approximately of 30 nm. Nonetheless, there is a wide spectrum of aggregate sizes in the population with an estimated standard deviation of 35%.

Other superparamagnetic nanoparticles, which are especially suitable to be used as positive contrast agents for magnetic resonance imaging, are those disclosed in Taboada et al., “Relaxometric and Magnetic Characterization of Ultrasmall Iron Oxide Nanoparticles with High Magnetization. Evaluation as Potential T1 Magnetic Resonance Imaging Contrast Agents for Molecular Imaging”, Langmuir 2007, Vol. 23, pp. 4583-4588. The nanoparticles have a core of maghemite and are citrate-coated. They are manufactured by decomposition of iron pentacarbonyl in the presence of oleic acid in an organic solvent, and further precipitated with ethanol and stabilized in hexane. The maghemite nanoparticles are then stabilized in an aqueous system by displacing the steric surfactant with tetramethylammonium hydroxide and precipitated to be resuspended in water. Further, a solution of citrate sodium is added while bringing the pH to neutral by adding dropwise 0.1 M of HNO₃. The nanoparticles so obtained have a mean particle diameter of 4.8 nm, with a standard deviation of 12%. When Taboada et al. measured the hydrodynamic diameter of three samples (Fe₂O₃-hexane, Fe₂O₃-water and Fe₂O₃-citrate) the resulting hydrodynamic diameters were, respectively, of 12±2 nm, 8±2 nm and 18±4 nm. They interpreted that the increase in size for Fe₂O₃-citrate was due to the incorporation of citrate anions at the surface of the iron particles, which may induce some aggregation between particles. Taboada et al. also hypothesises that these nanoparticles could be used as molecular imaging for cell tracking after the functionalization with specific biological ligands.

When nanoparticles are intended to be used embedded into the cell cytoplasm, it has been observed that the iron concentration is a critical point, since great amounts of iron are toxic for the cells due to the excess of the metal in ferrous state being free to enter reactions generating free radicals.

Another problem associated to the synthesis of the nanoparticles in order to be used in the organism is that in biological fluids they tend to aggregate and the population of aggregate sizes can be widely distributed, thus leading sometimes to non-reproducible, failed or non-concluding assays when used in biological systems, such as when they are endocytosed into the cells.

According to the prior art, and with the aim to recover the brain injured areas after a stroke episode or ischemia, there is the need of new superparamagnetic particle labels which do not negatively affect the biological behaviour of the cells tagged with them and which allow the cells to be detected, conducted and retained in injured brain areas or in the surrounding areas of the same in order to exert its biological action (i.e., to support the re-endothelialization and neovascularization of injured brain endothelium).

### SUMMARY OF THE INVENTION

The inventors have surprisingly found that endothelial progenitor cells loaded with superparamagnetic particles having specific homogeneous hydrodynamic diameter ranges, namely micro and nanoparticles, can migrate through the vascular system and reach the peripheral area of injured cerebrovascular tissue when directed by an external magnetic field. The cells are viable and maintain all their functionality, thus being able to secrete the growing factors essential to potentiate angiogenesis for the neo-vascularization and regeneration of injured tissue.

Then, in a first aspect the invention relates to an endothelial progenitor cell (EPC) comprising superparamagnetic iron oxide particles with an hydrodynamic diameter measured by dynamic light scattering comprised between 3 nm and 10 μm, for use in the treatment of cerebrovascular disorders or accidents, also named stroke.

This first aspect of the invention could also be formulated as the use of an endothelial progenitor cell (EPC) comprising superparamagnetic iron oxide particles with an hydrodynamic diameter measured by dynamic light scattering comprised between 3 nm and 10 μm, for the preparation of a medicament for the therapy of cerebrovascular disorders or accidents, also named stroke. In other words and a more concrete manner, the invention elicits a method of treatment of cerebrovascular disorders or accidents, in which a subject suffering from said cerebrovascular disorders or accidents, also named stroke, is treated with a therapeutically effective amount of an endothelial progenitor cell (EPC) comprising superparamagnetic iron oxide particles with an hydrodynamic diameter measured by dynamic light scattering comprised between 3 nm and 10 μm.

Contrary to the prior art teachings indicating that the loading of EPC with superparamagnetic particles could impair their migration through the vascular system (Yang et al., supra), the inventors surprisingly found that these kind of cells when loaded with particles of specific sizes and homogeneous hydrodynamic diameter, not only were able to migrate from the application area (tail vein) through all the systemic vascular system, but also could be retained around the brain injured area which could improve the therapy of cerebrovascular accidents, such as stroke or ischemia of any aetiology (trauma, emboli, tumour, etc.). Yang et al. (supra) labelled swine peripheral blood-derived EPC and rat bone marrow-derived EPC with SPIO nanoparticles alone and found that SPIO nanoparticles without transfection agents (which allow to obtain sufficient intracellular SPIO nanoparticles for efficient MRI) could label EPC efficiently. However migration assays performed by Yang et al. with the cells demonstrated that SPIO nanoparticles impaired EPC migration ability and promoted EPC adhesion capacity.

The fact that the EPC of the invention can migrate through the bloodstream and be functional is surprising, since the cerebrovascular system is a particular one due to the very low permeability and high selectivity of the brain blood barrier.

Moreover, the approach proposed by the inventors represents a real advantage over the prior art because it attempts to solve the real cause of stroke (cerebrovascular accident), which is the interruption of blood flow. The promotion of the regeneration or new formation of the vascular tissue allows reconstitution of the brain damaged area from the surrounding damaged area. On the contrary, the model of Song et al. (supra) the infracted area is reduced by the provision of neural cells, but these cells will probably die and rest non-functional if the blood flow is not re-established.

No previous assays have been reported using EPC cells containing superparamagnetic particles and being able to migrate under a magnetic field in the brain, the cells being remotely injected in an organism with injured brain vasculature, and magnetically retained in the injured area by means of external magnetic fields. This remote application avoids the injection in the injured brain, which is severely compromised and allows a safety and pleasant administration for the patient.

In a second aspect, the invention relates to a particular superparamagnetic nanoparticle obtainable by the process comprising the following steps:

a) mixing in an inert atmosphere an organic iron precursor selected from the group consisting of iron acetylacetonate and iron pentacarbonyl with 1,2-hexadecanediol and a surfactant system in an organic solvent and refluxing the mixture at a temperature comprised between 270° C. and 350° C. for a period of time from 0.5 to 2.0 hours to obtain a superparamagnetic iron oxide black precipitate;
b) dissolving the precipitate of step a) in an organic solvent in the presence of the surfactant system to obtain a dispersion of superparamagnetic iron oxide nanoparticles with a coating comprising the said surfactant system;
c) to add while stirring an aqueous solution of the electrolyte tetramethylammonium hydroxide to the nanoparticle dispersion of step b);
d) to adjust the pH of the dispersion between 8 and 10 by adding dropwise with drop sizes from 25 to 200 μl and in a time span from 30 seconds to 3 minutes, a 0.01 M aqueous solution of an inorganic acid to form nanoparticles having a hydrodynamic diameter comprised between 10 nm and 100 nm measured by the Dynamic Light Scattering technique, said nanoparticles consisting in a core of an aggregate of sub-particles of superparamagnetic iron oxide and a coating of the electrolyte tetramethylammonium hydroxide;
e) to add an aqueous solution of a citrate salt to a final concentration of citrate molecules per nm² of the nanoparticle surface, N, comprised between 0.5 to 40, said nanoparticle surface determined as 4π(r_{aggregate of sub-particles)}², wherein r is the hydrodynamic radium of the aggregate sub-particle; and
f) further adjusting the solution of step e) to a pH comprised between 6.5 and 7.5 by adding, dropwise with drop sizes from 25 to 200 μl, a 0.01 M aqueous solution of an inorganic acid.

The nanoparticle so obtained can also be defined as a superparamagnetic iron oxide nanoparticle comprising a core of an aggregate of sub-particles of superparamagnetic iron oxide and a coating comprising citrate, said superparamagnetic iron oxide nanoparticle having a hydrodynamic diameter comprised between 10 nm and 100 nm.

A variant of the process to obtain the superparamagnetic nanoparticles includes the starting step a) of mixing in an inert atmosphere an organic iron precursor selected from the group consisting of iron acetylacetonate and iron acetate with a surfactant system in an organic solvent and then heating the mixture by microwave at a temperature comprised between 150° C. and 180° C. for a period of time from 5 minutes to 0.5 hours. This variant also allows the obtention of a black precipitated product. Further, steps b) to f) are performed.

This variant results of special interest since it avoids refluxing temperatures and may be performed in a short time.

Independently of the first step of the method, the method allows controlling the nanoparticle size, namely the hydrodynamic diameter of the aggregate of sub-particles constituting the core of the nanoparticles, thus obtaining a homogeneous population of nanoparticles, apart of having also a homogeneous population of sub-particle aggregates. Surprisingly, said homogeneous nanoparticles, in dispersion form, may be efficiently internalized by the EPC, thus allowing a concentration of iron inside the cell comprised between 20 pg/cell and 40 pg/cell. Preferred values are around 30 pg/cell. This concentration is substantially greater than the prior art values (around 12 pg/cell). Iron content refers to amount of iron ions and is determined as disclosed in Kyrtatos et al., “Magnetic Tagging Increases Delivery of Circulating Progenitors in Vascular Injury”, J. Am. Coll. Cardiol. Intv. 2009, Vol. 2, pp.: 794-802.

Greater amounts of iron allow the maintenance of the same in spite of the subsequent cell division cycles. Contrary to what would be expected, these iron concentrations are not toxic for the EPC cells. The cells can migrate through the vascular system and get the cerebrovascular system, where they are retained, especially well if an external magnetic field is applied, and can promote regeneration of endothelization (vascularization) by secreting the specific growing factors. Moreover and unexpectedly, the cells are able to secrete higher amounts of endothelial factors, namely VEGF, than those cells without the nanoparticles, namely without the aggregates of nanometric size.

The sub-particle aggregates obtainable as above disclosed present a dendritic perimeter or form. It is suspected that this particular outline may contribute to the good internalization observed in EPC cells.

Without being bound to the theory, it seems that the biocompatibility anionic surface charge of the surface functionalization, the homogeneity of the sub-particle aggregate size, the specific selected values of hydrodynamic diameter, and are the responsible of this non-toxicity and higher activity. It is postulated that the negatively charged citrate surface and the absence of any polymer provide optimum type of interactions with the plasma membrane.

The nanoparticles of the invention, with a homogeneous size population, are suitable to be used in cell labelling without disturbing the cell viability and functionality. Moreover, great amounts of nanoparticles may be loaded (endocytosed) into EPCs without impairing the latter and making them long time useful in spite of the cell division that tends to dilute the superparamagnetic labelling or load.

In a third aspect, the invention relates to an endothelial progenitor cell comprising the superparamagnetic nanoparticles as defined above. Thus, an endothelial progenitor cell comprising a superparamagnetic iron oxide nanoparticle, wherein said nanoparticle comprises a core of an aggregate of sub-particles of superparamagnetic iron oxide and a coating comprising citrate, and wherein said superparamagnetic iron oxide nanoparticle has a hydrodynamic diameter comprised between 10 and 25 nm.

As above exposed, these cells may accept great amounts of iron, probably due to the nature of the nanoparticles, without toxicity effects. These EPCs secrete great amounts of VEGF that may accelerate the vascular regeneration or neo-vascularization in respect of the cells without the nanoparticles.

Finally, a fourth aspect of the invention is a pharmaceutical composition comprising the nanoparticles as defined above or an EPC cell containing the said nanoparticles and any pharmaceutically acceptable excipients.

### BRIEF DESCRIPTION OF THE DRAWINGS

FIG. 1 shows magnetic resonance imaging (MRI) images (panels A and B) of a mouse brain in which an infarct was provoked at cortical level by occlusion of middle cerebral artery. Hyperdense (white) area corresponds to damaged tissue. Arrows show hypodense black spots corresponding to the accumulation of EPC loaded with superparamagnetic microparticles. The image also shows a major concentration of nanoparticle-labeled EPCs under the influence zone of the magnet (indicated as a dashed-line square in panel A).

FIG. 2 is a microscope image of the bottom transwell (BT) and bottom plate (BP) of a transwell chamber. EPCs: endothelial progenitor cells; EPC-NP: endothelial progenitor cells loaded with nanoparticle aggregates; EPC-NP+MAGNET: endothelial progenitor cells loaded with nanoparticle aggregates under the influence of a magnetic field.

FIG. 3 Phantom images of the superparamagnetic nanoparticle aggregates are shown at different concentrations. A is agarose, T2WI (in panel A) is the relaxivity time 2 weighted image, T1WI (in panel B) is the relaxivity time 1 weighted image.

FIG. 4 is a TEM image of the superparamagnetic nanoparticles of the invention.

FIG. 5 is a phantom image of increasing number of EPCs loaded with 50 micrograms/mL of the superparamagnetic nanoparticle aggregates of the invention. A is agarose, T2WI is the relaxivity time 2 weighted image. Hypodense black spots correspond to nanoparticle-loaded EPCs.

FIG. 6 is an optical microscope image (Olympus IX71) of the vessel structures performed by EPCs without nanoparticles (EPC; panel (A)) and with EPCs loaded with citrate-coated SPIO nanoparticles (EPC-NP; panel (B)).

FIG. 7 is a graphic showing the number of rings per analysed field (panel (A)), the perimeter of the vessel structures (panel (B)) and the observed cell junctions (panel (C)) within EPCs of the invention (EPCs-NP) in respect of a control (EPCs) visualized by optical microscope.

FIG. 8 shows magnetic resonance imaging (MRI) images (panels A, B and C) of a mouse brain to which EPCs of the invention were injected. Arrows show hypodense black spots corresponding to the accumulation of EPC loaded with superparamagnetic nanoparticles. The image also shows a major concentration of nanoparticle-labeled EPCs (EPC-NP) within the influence zone of the magnet (indicated as a dashed-line square).

FIG. 9 is a microscope image of a Prussian Blue staining of the brain tissue. Allows visualizing the presence of magnetized EPCs in brain cortex under the influence of a magnetic field (Panels A and B), or unexposed to any magnetic field (Panel C). Arrows indicate areas with the blue-stained cells.

### DETAILED DESCRIPTION OF THE INVENTION

As will be illustrated below, endothelial progenitor cells (EPC) loaded with superparamagnetic particles, said particles having an hydrodynamic diameter from 5 nm to 10 μm, measured by DLS, and being homogeneous in size according a standard deviation which is lower than 60% may be for use in the treatment of cerebrovascular disorders. In other words, EPC are used for the preparation of a medicament for the treatment of an acute cerebrovascular disorder, also named stroke. This effect is due to the ability of the cells to specifically reach the injured area or its peripheral zone and to continue in performing its function when sited in these areas. The effect is improved under the influence of an external magnetic field.

For the purpose of understanding the following definitions are included:

The “particles”, as defined in the present invention, represent a unitary entity with a specific size and they may consist in a unitary body or in an aggregation of smaller particles (sub-particles), said aggregation constituting as a whole a single and differentiated body also. The nature of the particles is normally dependant on the nature of the materials performing them and the environment wherein they are disposed. With this sense, the invention defines also as particles those which, in turn, are made of sub-particles physically contacted or connected (van der Waals forces, electrostatic attraction, etc.) and presented as a unitary entity, clearly differentiated from the medium wherein they are sited (suspended).

The expression of “aggregate of sub-particles” means the sum of several sub-particles which as a whole constitute a unitary entity (i.e. the “particle”) with a determined size.

The expression “endothelial progenitor cells comprising/loaded with superparamagnetic particles” means that the cells include the particles in any of the organules or vesicles of the cytoplasm of the cell, as well as in free form in the cytoplasm. The loading of the cells is normally performed by promoting an endocytic pathway, similar to those used for internalizing some drugs or cell factors, receptor mediated or non-receptor mediated.

The term “hydrodynamic diameter” means the diameter determined by dynamic light scattering (DLS) techniques based on the Stoke-Einstein equation. It does it by illuminating the particles with a laser and analysing the intensity fluctuations in the scattered light. Dynamic light scattering measures Brownian motion and relates it to the size of the particles for which light intensity is proportional to the square of the volume of the particle. The resulting diameter corresponds not only to the particle diameter, but also to the molecules strongly adsorbed onto its surface. Therefore, the hydrodynamic size is always larger than the size observed by transmission electron microscopy.

The measurements of hydrodynamic sizes by Dynamic Light Scattering (DLS) in the present invention were performed with a Zetasizer Nano ZS from Malvern Instruments, equipped with a He/Ne 633 nm laser. The titration (measuring particle size while lowering the pH) was performed with the MPT-2 Autotitrator attachment. To evaluate the hydrodynamic size each sample was measured 3 times and each measurement was in turn composed of 7 to 20 runs. An average value was obtained. For titration, only one measurement was made at each pH.

In the sense of the invention “an electrostatic coating” is a cover or layer extended on the nanoparticle core formed by charged molecules, which promotes the colloidal stability via electrostatic repulsion between particles. It is a kinetic stabilization method not applicable to electrolyte sensitive systems and the stability of electrostatically stabilized colloids highly depends on pH.

In a preferred embodiment the endothelial progenitor cell (EPC) of the invention for use in the treatment of cerebrovascular disorders or accidents, comprises, preferably, superparamagnetic particles with an homogeneous hydrodynamic diameter, measured by dynamic light scattering (DLS), comprised between 3 nm and 10 μm, preferably between 5 nm and 10 μm, said homogeneity determined as the deviation standard of the particle population size and which is lower than 60%.

In a more preferred embodiment the particles loaded into the EPC have a hydrodynamic diameter from 3 nm to 5 μm, preferably from 5 nm to 5 μm.

In another preferred embodiment, the standard deviation is lower than 50%, preferably comprised between 5% and 50%, and more preferably lower than 40%, preferably between 10% and 40%.

In another preferred embodiment the particle aggregates loaded into the EPC have a hydrodynamic diameter range from 10 nm to 100 nm. Preferred hydrodynamic diameters range from 15 nm to 60 nm. Still more preferred hydrodynamic diameters are comprised between 10 nm and 25 nm. In a particular embodiment the hydrodynamic diameters are comprised between 10 nm and 25 nm, being preferred the hydrodynamic diameters comprised between 10 nm and 15 nm.

In a more preferred embodiment, the particles consist in an aggregate of sub-particles.

Preferred particles of the EPC for use in the treatment of stroke consist in sub-particle aggregates that comprise a core of magnetite and/or maghemite (as iron oxide compound) and a coating comprising at least molecules with negative charge. The preferred coatings are those comprising a citrate salt, tetramethylammonium hydroxide (TMAOH) or mixtures thereof.

It is a preferred embodiment a superparamagnetic nanoparticle obtainable by the process as disclosed above in which the surfactant system of step a) comprises oleic acid, oleylamine, or a mixture thereof.

In another preferred embodiment, the organic solvent of steps a) and b) is selected from the group consisting of hexane, toluene, heptane, benzene, and mixtures thereof.

A preferred superparamagnetic nanoparticle is obtainable by using a citrate salt selected from the group consisting of sodium citrate and potassium citrate.

In a preferred embodiment the superparamagnetic nanoparticle has a hydrodynamic diameter comprised between 15 nm and 60 nm. Still more preferred hydrodynamic diameters are comprised between 10 nm and 25 nm. In a particular embodiment the hydrodynamic diameters are comprised between 10 nm and 20 nm, being preferred the hydrodynamic diameters comprised between 10 nm and 15 nm.

Preferred inorganic acids used to adjust the pH of the solutions of step d) and f) include nitric acid and hydrochloride acid, being most preferred the nitric acid.

In a preferred embodiment, the superparamagnetic nanoparticle is obtainable by a process comprising steps a) to f) as above indicated and further including purification steps between step b) and c), namely including the precipitation of the dispersion with a (C1-C4)-alcohol solution such as ethanol, methanol, isopropanol and, after removing the solvent, if needed with the aid of a centrifuge, to disperse the nanoparticles in an organic solvent to obtain a black-brown nanoparticle dispersion in said organic solvent.

In a preferred embodiment, the superparamagnetic nanoparticle is obtainable by a process consisting in all steps a) to f) as above indicated.

In a most preferred embodiment, the superparamagnetic nanoparticle is obtainable by a process comprising the following steps:

a) mixing iron acetylacetonate, with 1,2-hexadecanediol with a surfactant system including oleic acid and oleylamine in benzyl ether, and heating the mixture to a temperature comprised between 180° C. and 250° C., for a period of time from 1.5-2.5 hours;
b) refluxing the mixture of step a) at a temperature comprised between 270° C. and 350° C. for a period of time from 0.5 to 2.0 hours, to obtain a superparamagnetic iron oxide black precipitate in form of magnetite, maghemite or mixtures thereof;
c) leaving at room temperature (18° C.-25° C.) the precipitate of step b) and dissolving it in hexane in the presence of oleic acid and oleylamine to obtain a dispersion of superparamagnetic iron nanoparticles with a coating comprising oleic acid and oleylamine;
d) to precipitate the dispersion obtained in step c) with ethanol, and after removing the solvent to disperse the nanoparticles in hexane to obtain a black-brown nanoparticle dispersion;
e) to add while stirring an aqueous solution TMAOH, preferably with a concentration comprised between 10% and 20% by weight, to the nanoparticle dispersion of step d), thus obtaining nanoparticles with an electrostatic coating of TMAOH;
f) to precipitate the nanoparticles of step e) with acetone;
g) mixing the precipitate of step f) with an aqueous solution of the TMAOH with a concentration comprised between 0.01% and 10% by weight, resulting in a nanoparticle dispersion with a pH comprised between 10 and 12, and further to centrifuge the said dispersion at 4000 rpm during 10 min to collect the supernatant; particularly the concentration of the TMAOH is comprised between 1.0% and 10% by weight, and preferably the concentration of the TMAOH is comprised between 0.01% and 0.1% by weight;
h) to adjust the supernatant solution of step g) to a pH comprised between 8 and 10 by adding dropwise with drop sizes from 25 to 200 μl and in a time span from 30 seconds to 3 minutes, a 0.01 M aqueous solution of an nitric acid to form nanoparticles having an hydrodynamic diameter comprised between 10 nm and 100 nm measured by the Dynamic Light Scattering technique, said nanoparticles consisting in a core of an aggregate of sub-particles of superparamagnetic iron oxide magnetite, maghemite or mixtures thereof, and a coating of the electrolyte tetramethylammonium hydroxide;
i) to add an aqueous solution of a sodium citrate to a final concentration of citrate molecules per nm² of the nanoparticle surface, N, comprised between 0.5 to 40 molecules/nm², said nanoparticle surface determined as 4π(r_{aggregate of sub-particles)}², wherein r is the radium of the aggregate of sub-particles; and
j) further adjusting the solution of step i) to a pH comprised between 6.5 and 7.5 by adding, dropwise with drop sizes from 25 to 200 μl, a 0.01 M aqueous solution of nitric acid.

The control of the nanoparticle size is performed by the fine regulation of the pH gradient. Regarding the initial solution (that obtained in step g)), the more alkaline the smaller the aggregate of sub-particles, and thus the smaller nanoparticle obtained. When acid is added pH decreases and the aggregate size increases (i.e.: from a nanoparticle of 15 nm at pH=13, a nanoparticle of 30 nm can be obtained when lowering to pH=10, and of 40 nm if the pH is adjusted to 8). Afterwards, the addition of a citrate solution (pKa₃=6.40) allows further addition of an acid to reach the physiological pH (6.5-7.5) without altering the nanoparticle size.

In a preferred embodiment, the process for obtaining superparamagnetic nanoparticle is performed starting by mixing at room temperature the organic iron precursor and the surfactant system in an organic solvent, followed by microwave heating. Preferred heating power is 200 W, a preferred heating temperature is 170° C., and a preferred time of heating is 5 minutes. Alternatively, a preferred temperature is 160° C. and a preferred time of heating is 15 minutes.

A pH of 13 for the suspension obtained in step g) is reached by adding 15 μl of concentrated TMAOH (25% by weight) to 3 ml of water. The final TMAOH solution, which corresponds to a solution of TMAOH at 7% by weight is then added to the nanoparticle precipitate of step f).

The invention provides also endothelial progenitor cells with improved properties. In a preferred embodiment these cells comprise an iron concentration comprised between 1 pg Fe/cell to 40 pg Fe/cell. In a most preferred embodiment the iron concentration is comprised between 20 pg Fe/cell to 40 pg Fe/cell. This iron concentration can be determined in early EPC. As indicated below in the examples, with the cell culture proceedings two types of EPCs are obtained: an EPC-enriched population at day 5-6 after seeding (named early EPCs) and outgrowth EPCs which appear after several weeks of culture as a growing colony displaying endothelial morphology and features with clonogenic capacity. In another most preferred embodiment, the iron concentration is comprised between 1 pg Fe/cell to 20 pg Fe/cell, which can be preferably determined in outgrowth EPCs according to the invention.

The determination of the iron content is performed by treating the cells during 24 h with a solution of nanoparticles comprising from 25 to 100 μg Fe/ml. Then the quantity of iron/cell is calculated from the comparison of the remanence magnetization value at −263° C. of about 200,000 cells containing nanoparticles and the remanence magnetization value obtained from a known mass of the superparamagnetic iron oxides. This analytical method is disclosed in Kyrtatos, P. G., et al., (2009) “Magnetic Tagging Increases Delivery of Circulating Progenitors in Vascular Injury”, J. Am. Coll. Cardiol. Intv.—2009, Vol. 2, pp. 794-802.

The EPCs of the invention are obtained by co-incubation of the particles (nanoparticle aggregates or microparticles) and the cells. Functionality and the use of the cells according to the invention are not compromised for the cell type used, either early EPCs or outgrowth EPCs.

In a preferred embodiment the endothelial progenitor cell comprises a superparamagnetic iron oxide nanoparticle, said nanoparticle consisting in a core of an aggregate of sub-particles of superparamagnetic iron oxide and a coating comprising citrate, said superparamagnetic iron oxide nanoparticle further having a hydrodynamic diameter comprised between 10 and 25 nm.

In a preferred embodiment, the endothelial progenitor cell of the invention comprises a superparamagnetic iron oxide nanoparticle that has a hydrodynamic diameter comprised between 10 and 20 nm. More preferably, the hydrodynamic diameter is comprised between 10 and 15 nm.

In a particular embodiment, the endothelial progenitor cell comprises a superparamagnetic iron oxide nanoparticle, said nanoparticle consisting in a core of an aggregate of sub-particles of superparamagnetic iron oxide and a coating comprising citrate and TMAOH.

With the EPCs of the invention pharmaceutical compositions, preferably injectable pharmaceutical compositions can be performed. The pharmaceutical compositions are injected for example through a limb vein (i.e.: tail vein in animals) and then the cells contained in them are let to reach the injured brain area. In order to improve the effect of the cells, it has been observed that they can be lead by means of an external magnet sited near the injured brain area, with the aim of retaining at this healthy area the maximum of these effective cells as will be illustrated bellow in the Examples. As example of usable magnet, used in the examples below is a permanent magnet of NdFeB (Nd2Fe14B) of aprox. 0.3 Tesla with dimensions 4×3×2 mm³.

Throughout the description and claims the word “comprise” and variations of the word, are not intended to exclude other technical features, additives, components, or steps. Furthermore the word “comprise” and its variations encompasses the expression “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

### EXAMPLES

### Example 1

### Synthesis of Superparamagnetic Nanoparticles (Sub-Particle Aggregates with a Coating of Citrate)

The method chosen to synthesize the nanoparticles was based on the method of Sun et al. “Monodisperse MFe₂O₄ (M=Fe, Co, Mn) Nanoparticles” J. Am. Chem. Soc.—2004, Vol. 126, pp. 273-279. The process involves high-temperature decomposition of iron acetylacetonate at 200° C., followed by the reaction with 1,2-hexadecanediol at 300° C. to form inverse spinel iron oxide nanoparticles. Once formed, the NPs are stabilized with oleic acid and oleylamine which bond sterically to the iron oxide surface controlling the size and preventing agglomeration of the NPs.

Experimental procedure: Iron (III) acetylacetonate (2 mmol, 0.71 g), 1,2-hexadecanediol (10 mmol, 2.584 g), oleic acid (6 mmol, 1.9 mL), oleylamine (6 mmol, 1.97 mL), and benzyl ether (20 mL) were added to a three-neck flask. Reaction was performed under argon atmosphere. The flask was immersed into an oil bath over a heating plate. The oil was heated to 200° C. and was left at that temperature for 2 h. The oil has a boiling temperature of about 250° C., and therefore the oil bath/heating plate system was replaced with a preheated heating mantle to obtain a reflux temperature of 300° C. The flask was kept in the heating mantle for 1 h, and then the heating mantle was removed and the solution was left to cool down to ambient temperature. After the reaction the particles needed to be purified from solvent, any unreacted reactants and possible by-products. Therefore 40 mL of ethanol were added to precipitate the particles and the mixture was centrifuged for 20 min at 4000 rpm. The supernatant was decanted and discarded, and hexane was added to redisperse the particles. The nanoparticles were centrifuged in hexane. The supernatant was kept for further purification with ethanol.

Alternatively, the procedure to obtain nanoparticles may be started by dissolving Iron (III) acetylacetonate (0,675 mmol) and oleic acid (2.025 mmol) in benzyl alcohol (4.5 ml) at room temperature. Fe(acac)3, oleic acid and benzyl alcohol were purchased and used without further purification. The mixture turned from red to reddish brown. This precursor suspension was heated by microwave at 170° C. for 5 min and a black product was obtained. (See Idalia Bilecka et al. “Simultaneous formation of ferrite nanocrystals and deposition of thin films via microwave-assisted nonaqueous sol-gel process”, J. Sol-Gel Sci. Technol—2010. Vol 57, num 3, pp. 313-322 [Online early access]. DOI: 10.1007/s10971-010-2165-1). Then, the purification steps were performed (ethanol precipitation and hexane redispersion).

30 mg of the nanoparticles with a coating comprising oleic acid and oleylamine were dispersed in 3 mL hexane. Then, 3 mL of 0.4 M TMAOH solution was added, and the mixture was magnetically stirred for 18 hours. After that, acetone was added to the mixture to precipitate the particles. The sample was centrifuged for about 15-20 minutes at 4000 rpm, and the light yellow supernatant was discarded. The precipitated particles were then redispersed in 7.5 mL water and 20 μL TMAOH 25 wt % in water (percentage by weight in water), and it was centrifuged at 4000 rpm for 10 minutes to ensure that all the particles were stable in the TMAOH-solution. Any precipitation was discarded, and only the supernatant was used.

A dispersion of the nanoparticles with the electrostatic coating of TMAOH (18 mL) was added to a solution of sodium citrate (2 mM) in deionised water. The dispersion was magnetically agitated and the pH was lowered with a solution of nitric acid. The pH was determined by paper. The final concentration of sodium citrate molecules per nm² of the nanoparticle surface, N, was of 14 molecules/nm². The lowering of the pH was performed as follows: adding 500 microliters of 0.01 M nitric acid every 30 seconds and for 3 minutes.

The iron content in the sample was determined by Flame Atomic Absorption Spectrometry (FAAS) to be 0.8 mg Fe/mL (corresponding to 1.1 mg of magnetite/mL).

The sample was also analyzed with DLS one day after its preparation, and four weeks later. The aggregates constituting the nanoparticles ranged from about 10 to 100 nm (hydrodynamic diameter), the most of them being between 15 and 30 nm, the mean size being 24 nm with a standard deviation of 18%. Measurements taken 4 weeks later revealed a mean size of 25 nm, thus indicating the good stability of the aggregates.

As can be seen in FIG. 4, which is an electronic image (TEM) of the nanoparticles according to the invention, it can be seen that the particles (that form aggregate of sub-particles in solution) have a homogeneous size.

Transmission electron microscopy (TEM) images were obtained with different instruments; a JEOL JEM-1400 operating at 200 keV and a JEOL JEM-1210 operating at 120 keV. High resolution images (HRTEM) were taken with a JEOL JEM 1010 equipped with a high resolution digital camera. Electron diffraction was performed with the microscope JEOL JEM-1210 with 23.1 mm A camera length (L).

Moreover, the results of an assay performed in a superconducting quantum interference device (SQUID) at room temperature (18° C.-25° C.) indicated that the magnetic properties of the nanoparticles (aggregates of sub-particles of magnetite with a coating of citrate molecules) do not show remanence, as expected for a superparamagnetic material and saturate at relative small fields (<1 Tesla) and with a high saturation magnetization, which is advantageous for achieving large response to a magnetic field of moderated strength.

### Example 2

### EPC Culture and Labelling with Superparamagnetic Particles

### EPC Culture:

Male BALB/C mice, weighing 20-25 g, were obtained from Charles River Laboratories. Spleens were obtained after cervical dislocation and kept in cold Hank's Buffered Salt Solution (HBSS)+4% bovine serum albumin (BSA)+1 mM ethylendiamintetracetic acid (EDTA) buffer. Under the hood, spleens were minced using a scalpel and heated at 37° C. in EDTA (1 mM). Afterwards, new HBSS+4% BSA media was added and minced tissue was run thorough a 40 microns nylon filter to obtain single cell suspension. Mononuclear cell (MNC) separation was done by density gradient by the Ficoll method: 6 mL of ficoll and a top layer of the cell suspension centrifuged 20 minutes at 1500 rpm. The medium layer containing the MNCs was obtained. Remaining eritrocytes were removed by adding red blood cell lysis buffer (150 Mm NH4Cl, 10 mM NaHCO₃ and 0.1 mM EDTA in distilled water) and gently washed twice with complete growth media EGM-2 (Endothelial Cell Growth Media-2, Clonetics®, CA, USA) containing 10% fetal bovine serum (FBS), human endothelial growth factor (hEGF), vascular endothelial growth factor (VEGF), human fibroblast growth factor b (hFGF-B), insuline like growth factor 1 (R3-IGF-1), the antibiotic mixture GA-1000 (gentamicin and amphoterecin-B), heparin, hydrocortisone and ascorbic acid. MNCs (1×10⁷ cells/mL) were seeded in fibronectin-coated (10 μg/ml Sigma-Aldrich) 25 cm² flasks (in 5 mL) or in 12-well plates (in 1 mL) and incubated in a 5% CO₂ atmosphere at 37° C. First media change was done at day 2 and then changed every other day. Two types of EPCs are obtained: an EPC-enriched population at day 5-6 after seeding and outgrowth EPCs which appear after several weeks of culture as a growing colony displaying endothelial morphology and features.

### Iron Oxide Nanoparticles Treatment:

At day 5-6 the cells were washed twice with phosphate saline buffer (PBS) and SPIOs of Example 1 were added at corresponding concentrations (0, 25, 50 or 100) μg/mL or 1×10⁷ Dynabeads® for cell uptake during 18-24 hours. All treatments were done in duplicates by co-incubation of the cells and the SPIOs.

### Prussian Blue Staining

To ensure SPIOs uptake a specific stain for iron content was performed. Briefly, cells were washed with PBS, fixed with 4% paraformaldehide for 30 minutes and rinsed with PBS twice. Perl's solution containing 1% potassium ferrocyanide+1% HCl was added for 30 minutes. Afterwards cells were washed with distilled water twice and observed under the microscope. Cells containing SPIOs turn blue dose-dependently (data not showed).

### Example 3

### Cell Viability Assay of EPC Comprising Superparamagnetic Nanoparticles of Example 1

### Cell Viability Assay

Measurement of the reduction of 3-(4,5-dimethylthiazol-2-yl)2,5-diphenyl-tetrazolium bromide (MTT) to produce a dark blue formazan product was performed to assesses the integrity of mitochondrial function as a measure of cell viability of EPCs comprising the nanoparticles (aggregates of sub-particles with a coating) of Example 1. At day 5-6 cells plated in 12-well plates were washed twice with PBS and MTT reagent was added to basal media at final concentration 0.5 mg/mL (total 1 mL per well). After 2 hours of incubation at 37° C., the medium was replaced by dimethyl sulfoxide to solubilizing the formazan. The absorbance of this colored solution was quantified by spectrophotometry at 590 nm wavelength. Dimethyl sulfoxide absorbance was subtracted to all sample measurements to allow standardization, each sample was measured twice to obtain a mean value and results for treatments are expressed as a percentage of the control group absorbance (no SPIOs).

As shown in Table 1, a mean of cell viability from 90% to 100% was detected for all the assayed nanoparticle concentrations indicating a normal cell viability. Surprisingly, the cells endocytosed great amounts of iron (25 pg iron/cell), measures as indicated by Kyrtatos et al. (supra), without altering its viability. C in μg/ml is the concentration of nanoparticle put into contact with the cells, Viab (%) is the percentage of mean cell viability

TABLE 1
Percentage of cell viability

| | C (μg/ml) | | | | |
|---|---|---|---|---|---|
| | 0 | 25 | 50 | 100 | TOTAL |
| Viab. (%) | 100 | 102.50 | 93.75 | 108.34 | 100.63 |
| Nr. of | 5 | 4 | 4 | 3 | 16 |
| samples | | | | | |
| SD | — | 11.67 | 15.77 | 24.01 | 13.39 |

These data are indicative of normal cell viability at every of the nanoparticle concentration tested. Moreover, it is surprising that amounts greater than 50 μg/ml do not affect the cell survival. The advantage accompanying this fact is the possibility of loading the cells to make them more sensitive to external magnetic fields or to improve Magnetic Ressonance images.

### Example 4

### Viability Data and VEGF Secretion of EPC

### Growth Factors' Secretion:

To assess growth factors' secretion from EPCs before and after SPIOs uptake of Example 1 as an indirect measure of cell function, conditioned media were obtained. Briefly, at day 6 cells plated in 12-well plates were washed twice with PBS and fresh media was added with corresponding concentrations of SPIOs for 18-24 hours. Afterwards, cells were deeply washed with PBS and 1 mL of fresh basal media (without growth factors or FBS) was added per well. Twenty-four hours later the media (named conditioned media, CM) was collected and concentrated thorough centrifugation with a 10 kDa filtering membrane. Volume was reduced from 2 mL (1 mL per duplicate) to approximately 250 μL. Total protein content was measured for CM by the Bradford's method and growth factors (VEGF, FGF, PDGF-BB and HGF) by multiple ELISA (Searchlight® technology). Concentration of growth factors content in CM was corrected by total protein content.

Following Table 2 shows the concentration of VEGF in pg/pg of protein, secreted by EPCs of the invention (previously in contact with nanoparticle at a concentration of 25 and 50 μg/mL). The control was a culture of EPCs not previously in contact with the superparamagnetic nanoparticles of the invention (0 μg/mL).

TABLE 2

| EPC | VEGF concentration (pg/μg protein) |
|---|---|
| EPC (Control = 0 μg/mL) | 1.505 |
| EPC (25 μg/mL) | 4.467 |
| EPC (50 μg/mL) | 2.225 |

In an unexpected way, the endothelial progenitor cells of the invention, containing in the cytoplasm the superparamagnetic nanoparticles also object of the invention, not only were viable as depicted in Example 3, but also were able to secrete one of the growth factors used in the reparation of endothelial tissue in greater amounts than the same cells without the nanoparticles. These results suggest that when the cells loaded with the nanoparticles of the invention reach a cerebrovascular area surrounding an injured zone, they are enforced to promote neo-vascularization and reendothelization.

### Example 5

### EPC Migration (Transwell)

EPC migration was evaluated using a transwell chamber with a transwell pore of 8 μm (BD Falco, FluoroBlok).

Briefly, 3×10⁴ outgrowth EPCs were seeded in 12-well plates in EGM-2 complete media. After two days nanoparticles of Example 1 (NPs) were added overnight at 50 μg/ml to allow cellular uptake. NPs were not added for control cells. The day after transwells were coated with fibronectin. EPCs were trypsinized and labelled using the PKH26 red fluorescent cell linker (Sigma-Aldrich). Then 10,000 fluorescent cells were added in each transwell in EGM complete media and the same media was added to the bottom chamber. Three hours later the cells were attached to the transwell and the media was changed to basal media without g growth factors or serum.

Cells were allowed to migrate during 24 hours and for quantification 6 images were taken at 100× magnification in an inverted microscope (only the fluorescent cells which have migrated thorough the pores are visualized. Three conditions were tested: EPC, EPC-NP and EPC-NP with a neodymium magnet placed on the bottom of the lower chamber.

Following table 3 shows the number of cells migrating thorough the transwell pores.

| Condition | Number of EPCs (cell migration) |
|---|---|
| EPC | 396 |
| EPCs-NP | 478 |
| EPCs-NP + external MAGNET | 587 |

As can be seen in FIG. 2, the cells (EPC) loaded with the nanoparticles (EPCs-NP) and magnetically (EPCs-NP+MAGNET) assisted with an extern magnet reach the bottom of the transwell chamber (BP column). Thus, the cells with endocytosed nanoparticles can migrate through the vascular system, and are able to reach a zone of interest if externally managed by a magnet.

These results, together with those of Example 4 allow clearly to state that the cells are functional and may be used to be placed in an ischemic peripheral zone or stroke area in order to restore the damaged endothelial tissue.

### Example 6

### In Vivo Assay with Dynabeads®-Loaded EPC

Commercially available Dynabeads® of 4.5 μm diameter (Invitrogen) were used for in vivo cell tracking in a mouse model of cerebral ischemia.

Induction of ischemia was performed in mature male BALB/C mice, weighing 25-30 g (Charles River Laboratories). A reproducible model of stroke in the middle cerebral artery (MCA) affecting the cortex was induced by electro-cauterization of the distal portion of the left MCA. Body temperature was maintained at 36.5-37° C. using a heating blanket. The procedure was performed under isoflurane anesthesia (4% for induction and 2% for maintenance) via a face mask. Briefly, an incision was made between the left eye and ear and under an operating microscope (Leica MS5), the temporal muscle was divided exposing the left lateral aspect of the skull. The MCA was identified thorough the semi-translucent skull and a small burr hole was made using a high-speed microdrill at the level of the bifurcation of the M1 portion (at the level of the inferior cerebral vein). Saline was applied to the area throughout the procedure to prevent heat injury. Cerebral blood flow was measured continuously by laser-Doppler flowmetry using a flexible fiberoptic (Moorelab) placed directly on the top of the parietal branch of the M1 bifurcation beginning 5 minutes before MCAO, during and after the electro-cauterization. Using a micromanipulator holding a 30G needle (0.4 mm diameter) the MCA was compressed, a decrease in the cerebral blood flow was ensured and using a small vessel cauterizer the MCA was permanently occluded by indirect electrocoagulation thorough the 30G needle. Then, the muscle was replaced and the skin was closed using 5-0 silk suture and magnesic metamizol (400 mg/kg) was administered subcutaneously right after the procedure. The duration of anesthesia in all animals was <30 min. Mice that showed decreased cerebral blood flow by >75% after the procedure were used for experiments.

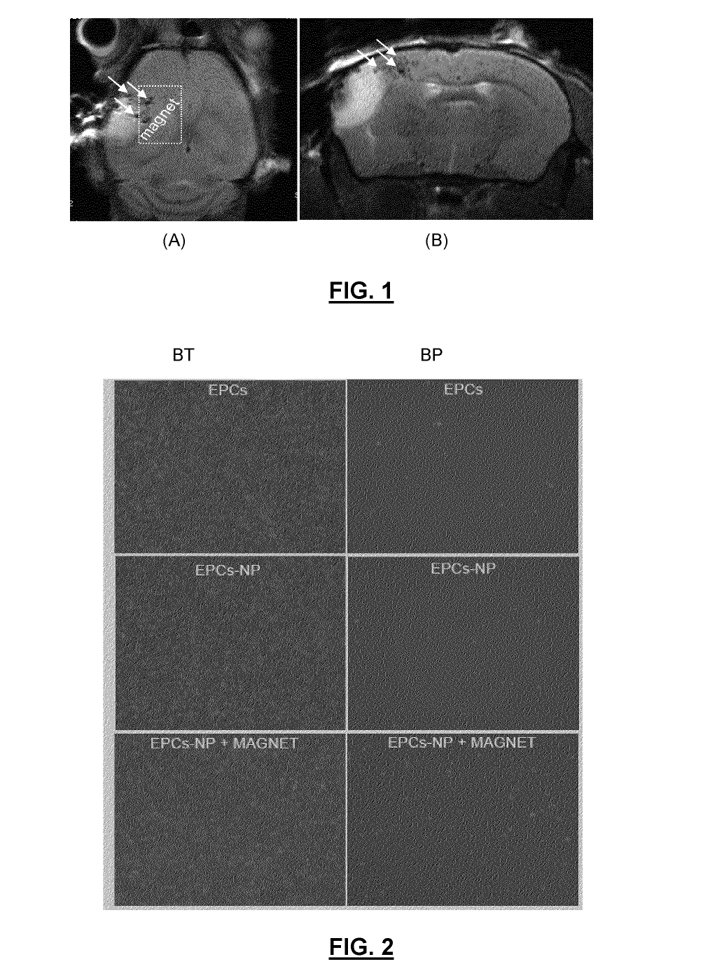

After surgery a magnetic field generated by a permanent magnet of NdFeB (Nd2Fe14B) of aprox. 0.3 Tesla with dimensions 4×3×2 mm³ was applied. The magnet was glued on the skull to retain the cells both in the injured tissue and the ischemic boundary corresponding to the middle cerebral artery and anterior cerebral artery territories respectively). Position of the magnet is represented in FIG. 1 with a dashed-lined square. Five hours after ischemia and magnet implantation a tail vein injection of 2.5×10⁵ EPCs labelled with Dynabeads® (example 2) was performed. MRI was performed 24 hours after ischemia.

The magnetic field applied to injured zone allow cells be retained in the ischemic peripheral zone (black spot of FIG. 1). Hyperdense white area of FIG. 1 is the zone with induced isquemia (or the stroke area). Thus, as revealed by the black spots, EPC cells remotely injected and comprising superparamagnetic particles with a diameter range from 5 nm to 10 μm are able to migrate to the peripheral injured zone, and be retained therein. Used Dynabeads had a diameter of 4.5 μm. Thus, the EPCs comprising the superparamagnetic particles represent a pool of putative mature endothelial cells prepared to restore the injured blood vessel/s.

### Example 7

### Magnetic Data of EPC with the Nanoparticles (Nanoparticles (NP) with a Core of Aggregated Sub-Particles and a Citrate Coating)

The MRI technique makes use of the signals produced by atoms with uneven number of protons when placed in a magnetic field. The most commonly atom used for MRI is the hydrogen atom, since it appears in the major components of the body: water and fat. When exposed to a strong magnetic field, B0, the spins of the protons align either parallel or antiparallel to the magnetic field. During their alignment, the spins precess around the magnetic field direction (around the z-axis) under a specified frequency, known as the Larmor frequency. They show a net magnetization in the z direction. When a radiofrequency (rf) pulse is introduced to the nuclei, these protons are perturbed. The protons absorb energy inducing a 90° flip, causing a decrease in magnetization along the z-axis and generating transverse magnetization in the xy-plane (Mxy), perpendicular to the direction of the static magnetic field. Upon removal of the RF pulse, the excited spins relax to their initial, lower-energy state, by recovering the net z magnetization (longitudinal or T1 relaxation, leading to T1 maps) and by losing the coherent spin motion in the x-y plane (transversal or T2 relaxation, leading to T2 maps or T2* maps). T2* maps consider the molecular interactions and local magnetic field non-uniformities. T2* value is always lower than T2 relaxation time. Image contrast is due to local variation in relaxation, and arises from proton density as well as the chemical and physical nature of the tissues within the specimen.

NPs Relaxation Properties (T2 and T1)

Agarose phantoms (1.5% in distilled water) containing increasing concentrations of superparamagnetic iron oxide nanoparticles (SPIOs) (0, 5, 10, 15 and 25 μg/mL) were prepared. Magnetic Ressonance Imaging (MRI) of phantoms was performed using a quadrature 7.2 cm inner diameter volume coil on a 7 Tesla Bruker BioSpec magnet and using the same geometry for all the scans: field of view (FOV)=8.5 cm×8.5 cm, 1.5 mm slice thickness with a 1 mm gap between slices (total 4 slices).

T2map, T1map and T2* were acquired to determine relaxation times using a multi-slice multi-echo (MSME) and variable repetition time (VTR) spin-echo and multi gradient echo (MGE) sequences respectively. Parameters for MSME (T2 maps) were: TR [repetition time]=3 s, 30 TE [echo time] values from 10 ms-300 ms (10 ms echo spacing), matrix size=128×128. Parameters for VTR sequence (T1 maps) were: TE=7.5 ms, seven TR values (1 s, 1.3 s, 1.7 s, 2 s, 2.6 s, 3.5 s, 5 s), matrix size=128×128. And parameters for multi-gradient echo MGE (T2* maps): TR=1500 ms, 30 TE values from 4 ms-300 ms (7 ms echo spacing), matrix size=128×128. Besides, High Resolution T2w images were acquired using Fast spin echo sequence RARE (rapid acquisition with relaxation enhancement): TR=4 s, TEeff [effective]=16 ms, 2 averages, matrix size=256×256. High Resolution T2*w images were acquired using gradient echo sequence FLASH (Fast low angle shot): TR=300 ms, TE=7 ms, flip angle=30°, 2 averages, matrix size=256×256. High resolution T1w images were acquired with gradient echo sequence: TE=4 ms, TR=250 ms, flip angle=50°, matrix size=256×256.

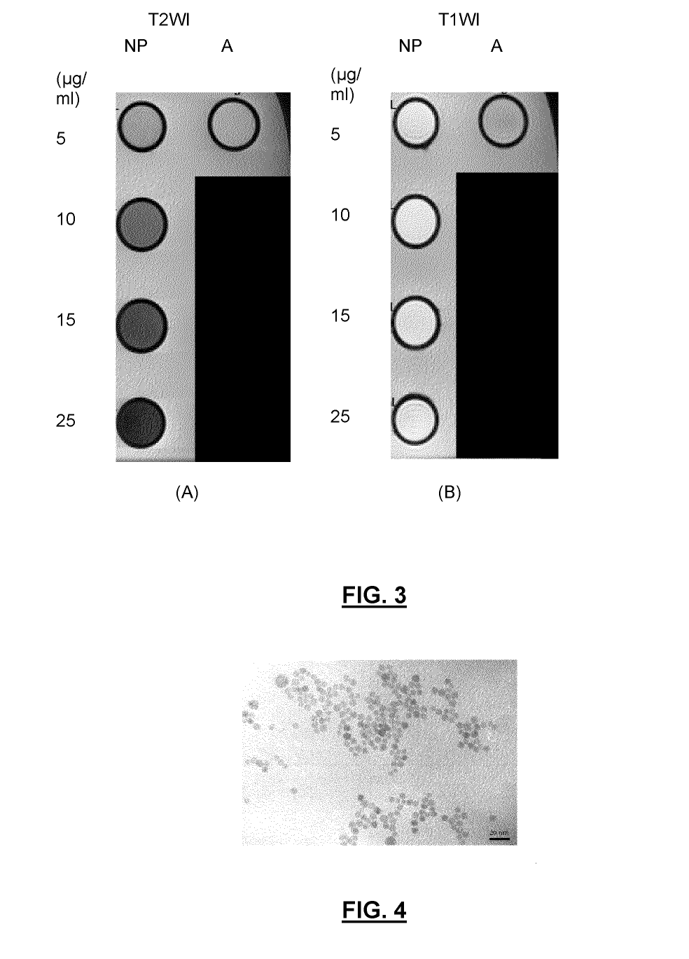

The image of the phantom obtained is shown in FIG. 3, panels (A) and (B). Concentrations of superparamagnetic iron oxide nanoparticles (SPIOs or NP) (5, 10, 15 and 25 pg/mL) are indicated beside the corresponding phantom cell and A indicates agarose phantom containing 0 μg/mL of SPIOs.

In Vitro Relaxation Properties of EPCs Labeled with NPs

EPCs were previously labeled with 50 pg/mL of nanoparticles during 18-24 h in culture media. Cell were collected by trypsinization and counted. Phantom cell suspensions containing increasing concentrations of cells (1.3×10³, 4×10³, 1.3×10⁴, 2.6×10⁴, 4.7×10⁴ cells) were prepared in 1.5% agarose gels. Magnetic Ressonance imaging of phantoms was performed using a quadrature 7.2 cm inner diameter volume coil on a 7 Tesla Bruker BioSpec and using same geometry for all the scans: field of view (FOV)=8.5 cm×8.5 cm, 1.5 mm slice thickness with a 1 mm gap between slices (total 4 slices).

T2map, T1map and T2* were acquired to determine relaxation times using a multi-slice multi-echo (MSME) and variable repetition time (VTR) spin-echo and multi gradient echo (MGE) sequences respectively. Parameters for MSME (T2 maps) were: TR=3 s, 30 TE values from 10 ms-300 ms (10 ms echo spacing), matrix size=128×128. Parameters for VTR sequence (T1 maps) were: TE=7.5 ms, seven TR values (1 s, 1.3 s, 1.7 s, 2 s, 2.6 s, 3.5 s, 5 s), matrix size=128×128. And parameters for multi-gradient echo MGE (T2* maps): TR=1500 ms, 30 TE values from 4 ms-300 ms (7 ms echo spacing), matrix size=128×128. Besides, High Resolution T2w images were acquired using Fast spin echo sequence RARE (rapid acquisition with relaxation enhancement): TR=4 s, TEeff=16 ms, 2 averages, matrix size=256×256. High Resolution T2*w images were acquired using gradient echo sequence FLASH (Fast low angle shot): TR=300 ms, TE=7 ms, flip angle=30°, 2 averages, matrix size=256×256. High resolution T1w images were acquired with gradient echo sequence: TE=4 ms, TR=250 ms, flip angle=50°, matrix size=256×256. Additionally, High Resolution T2w images were acquired using Fast spin echo sequence RARE (rapid acquisition with relaxation enhancement): TR=4 s, TEeff=100 ms, 2 averages, matrix size=256×256.

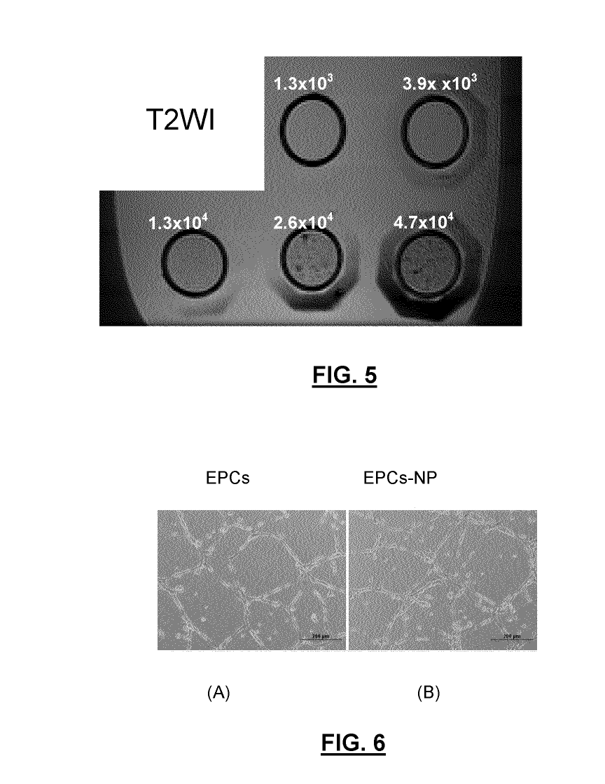

FIG. 5 shows the phantom image T2WI of the increasing concentrations of EPC loaded with nanoparticle aggregates (50 μg/mL) of the invention. Number of cells in 1.5% agarose are indicated beside each phantom.

The data retrieved from Example 7 allow to conclude that the nanoparticle aggregates are useful as contrast agents and may be used for cell-tracking.

### Example 8

### In Vitro Tubulogenesis Assay

Matrigel® (reduced growth factors, BD; San Jose, Calif., USA) was used as a substrate to assess tube formation capacity as an in vitro measurement of functional angiogenesis of mouse and human EPCs.

Briefly, tips and 24-well plates were placed at 4° C. for 2 hours. Afterwards, 200 microlitres of Matrigel® were carefully added onto each well and placed at 37° C. for 30 minutes. In the mean time outgrowth EPCs loaded with citrate-coated SPIOs (magnetized EPCs or EPCs-NP), said SPIOs nanoparticles with a hydrodynamic diameter of 24 nm with a standard deviation of 18%, were tripsinized. The same protocol was followed with non-loaded EPCs (non-magnetized). 6×10⁴ EPCs and EPCs-NP were seeded onto Matrigel-coated wells in 0.5 mL of basal media. Plates were incubated for 24 hours in a 5% CO₂ at 37° C. and images were taken at 10× (6 different fields). As can be seen in FIG. 6, which is an optical microscope image of the cells (Olympus IX71), vessel-like structures are formed by both loaded or EPCs-NP (panel B) and non-loaded EPCs (panel A). The number of complete rings, number of joints and perimeter with vessel-like structures were counted by personnel blinded for treatment. Greater amount of all the measured parameters are observed, surprisingly, when EPCs are loaded (or magnetized) with citrate coated SPIOs with a hydrodynamic diameter comprised between 10 and 25 nm, namely 24 nm.

These represent interesting data because they prove that the EPCs of the invention are much functional and effective than non-loaded EPCs.

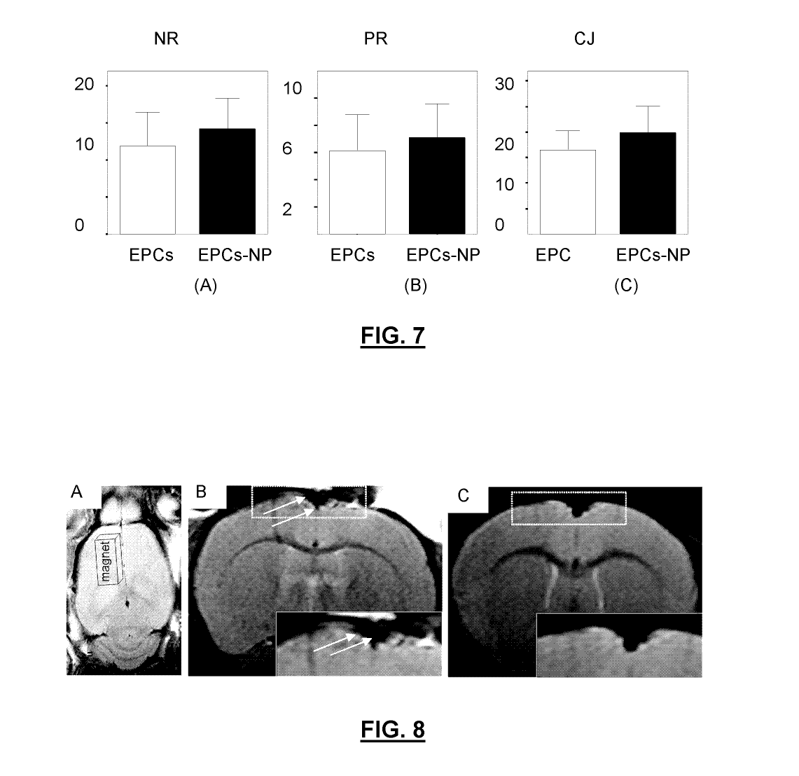

The same results are derivable from FIG. 7, wherein the number of rings (NR in panel (A)) per analysed field, the perimeter of the rings (PR in panel (B)) and the observed cell junctions (CJ in panel (C)) are represented in a diagram showing the values of EPCs-NP (that is EPCs loaded or magnetized with the citrate-coated SPIO nanoparticles, as well as the values for non-loaded EPCs (used as control).

The results are in addition summarized in following Table 4.

| | Number of | Perimeter of | Number of cell |
|---|---|---|---|
| | formed Rings | vessel structures | junctions |
| | (NR) | (PR) | (CJ) |
| EPCs | 11.8 ± 4.5 | 6.1 ± 2.6 | 16.5 ± 3.6 |
| EPCs-NPs | 14.1 ± 4.1 | 7 ± 2.5 | 19.8 ± 5.1 |

### Example 9

### Searchlight® Multiplex Protein Analysis

In a similar way than in Example 4, secretion of growth factors from mouse and human outgrowth EPCs was assessed as a measurement of cell functionality. Briefly, seeding density for EPCs was 4×10⁵ cells/flask (25 cm²) and grown for 2 days. Afterwards, cells were treated with 50 μg/mL of citrate-coated SPIOs (nanoparticles with a hydrodynamical diameter of 24 nm, SD 18%) for 24 hours in EGM-2 to allow EPC magnetization. To obtain conditioned media (CM), magnetized (EPCs-NP) and non-magnetized cells (EPCs) were deeply washed 3 times with basal media and 4 mL of fresh basal media were added to collect growth factors. At 24 hours the basal media containing EPCs' secreting factors (from EPCs and EPCs-NP), named conditioned media, was collected and concentrated using Amicon Ultra® centrifugal filters (Millipore, Ireland) with a 3 kDa membrane. Final conditioned media volume was around 600 microlitres and it was stored at −20° C. until use. Conditioned medias were assayed to determine the amount of EPC-secreted growth factors using the SearchLight® Human Angiogenesis Array 2 (Aushon Biosystems, MA, USA).

Following Table 5 shows the concentration of the secreted growth factors in pg/μg protein.

TABLE 5

| Growth | | | | |
|---|---|---|---|---|
| Factor | Mouse | Mouse | Human | Human |
| (pg/μg | Control | (Magnetized) | Control | (Magnetized) |
| protein) | EPCs | EPCs-NP | EPCs | EPCs-NP |
| FGF basic | 13.97 ± 5.89 | 23.23 ± 12.02 | 16.26 ± 4.48 | 25.60 ± 3.6 |
| VEGF | 0.22 ± 0.09 | 3.23 ± 2.34 | 10.08 ± 5.33 | 16.30 ± 4.64 |
| HGF | 0.43 ± 0.22 | 0.48 ± 0.19 | n.d. | n.d. |
| PDGB-bb | n.d. | n.d. | n.d. | n.d. |

Platelet derived growth factor (PDGF-bb),
hepatocyte growth factor (HGF),
fibroblast growth factor (FGF-basic) and
vascular endothelial growth factor (VEGF);
n.d.: non-detected.

Data from Table 5 clearly illustrate that, independently of the origin of the endothelial progenitor cells, when the cells are loaded with superparamagnetic iron oxide nanoparticles, they are able to produce higher amounts of several growth factors (VEGF and FGF basic) involved in the formation and regeneration of vascular tissue than the same cells without being loaded with such nanoparticles.

Thus, the endothelial progenitor cells of the invention are viable and highly functional to perform its tasks. Even, they are more functional than the corresponding cells without the nanoparticle load.

### Example 10

### In Vivo Assay with Citrated-Coated SPIO Nanoparticles Embedded in EPCs. In Vivo Functionality of EPCs-NP of the Invention

Following in vivo data confirm that magnetized EPCs of the invention can be successfully guided towards specific areas of the brain when an external magnetic field is applied.

Mice were anesthetized with isoflurane (4% induction, 1.5% maintenance) and 2 small magnets (3×4×2 mm) with a magnetic field of 0.3 T were implanted with glue in the left hemisphere and positioned as shown in FIG. 8 (left image). Afterwards, 3.5×10⁴ mouse early EPCs previously magnetized with 50 μg/mL of SPIO-NPs (EPCs-NP) were injected intravenously (tail vein) in 150 μL of PBS. Control animals received the same cell infusion without magnet implantation. Twenty hours later, magnets were removed and MR studies were carried out at 7 Tesla in a horizontal magnet (BioSpec 70/30 USR, Bruker BioSpin, Ettlingen, Germany) equipped with actively shielded gradients capable of 400 mT/m (B-GA12 gradient coil inserted into a B-GA20S gradient system) and a dedicated mouse brain quadrature receive surface coil, actively decoupled from a transmit volume coil with 72 mm inner diameter. Animals were positioned in the scanner bed, which allowed localized delivery of anesthesia (isoflurane, 0.5-1.5% in O₂ at 0.8 L/min; respiratory frequency monitored with a pressure probe and kept between 50-80 breaths/min). A recirculation water system, integrated in the animal bed, was used to control the body temperature as measured with a rectal probe (37° C.±1° C.). T2-weighted fast spin-echo images were initially obtained in axial, sagittal and coronal planes to be used as reference scout images for reproducible slice selection at each MRI session. Imaging parameters for these images were: echo time (TE)=12 ms, echo train length (ETL)=8, effective echo time (TEeff)=36 ms, repetition time (TR)=4s, field of view (FOV)=1.92×1.92 cm², matrix size (MTX)=128×128, and slice thickness (ST)=1 mm. Afterward, coronal MRI sections were performed over an 8.7 mm block starting 3.20 mm anterior to the bregma and towards the cerebellum. T2WI were acquired using a fast spin-echo sequence with ETL=8, TEeff/TR=36 ms/4.2 s, FOV=1.92×1.92 cm², MTX=256×256, 16 contiguous slices with ST=0.5 mm and 0.05 mm gap between them. T2 maps were also obtained using a multi-slice multi-echo sequence with 30 TE values ranging from 10 to 300 ms, TR=4 s, MTX=128×128, FOV=1.92×1.92 cm², and slices covering exactly the same brain region as in high resolution T2w coronal images but with 8 continuous 1-mm slices and 0.1-mm gaps.

Magnetic resonance imaging (MRI) depicted in FIG. 8 serves for showing in vivo brain cell tracking and guidance.

Panel (A) is a coronal section of a T2W image showing the position of a magnet implantation in the same way as for Example 6. Panels (B) and (C) are brain T2W images corresponding to mice injected with 3.5×10⁴ magnetized EPCs with (B) or without (C) magnet implantation. Inserts show areas under the influence of the magnetic field. Hypointense signal (black spots) are suggestive of the presence of magnetized EPCs. Arrows in panel (B) show hypodense black spots (area) corresponding to the accumulation of EPC loaded with the superparamagnetic nanoparticles (SPIO-NPs).

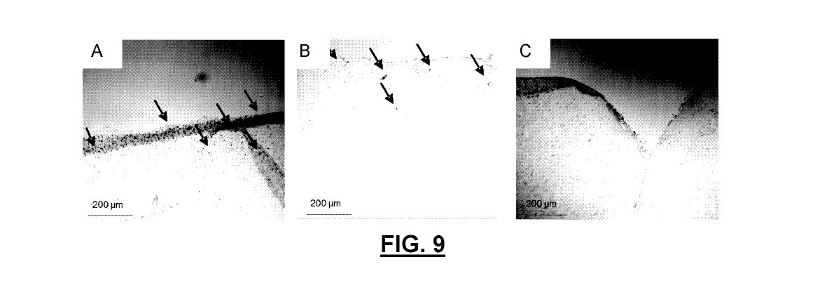

In addition a Prussian Blue staining of the brain tissue was performed in order to validate the MRI images. Stain confirms the presence of magnetized EPCs in brain cortex (signalized by means of arrows and visualizable as points in FIG. 9) under the influence of the magnetic field (Panels A and B) which were not seen in brains unexposed to the magnetic field (Panel C). Arrows indicate blue-stained cells.

FIGS. 8 and 9 allow concluding that endothelial progenitor cells of the invention, comprising citrate-coated nanoparticles of special size (EPC-NPs), can migrate through the bloodstream and be retained under a magnetic field disposed in a desired area of the brain (for example an injured area). In addition, the cells of the invention may be easily followed by MRI, which facilitates the task of the facultative in case of treatment of a patient. Also advantageously is that, once retained in the desired area, the cells of the invention will be functionally active, and will produce the growth factors needed to regenerate or to form vascular tissue allowing thus brain areas be properly irrigated. This functionality is even higher than the cells without being magnetized (as deduced from Example 9).

### REFERENCES CITED IN THE APPLICATION

Gazeau et al., “Magnetic labelling, imaging and manipulation of endothelial progenitor cells using iron oxide nanoparticles”, Future Medical Chemistry 2010, Vol. 2(3), pp. 397-408.
Yang et al., “Superparamagnetic iron oxide nanoparticles may affect endothelial progenitor cell migration ability and adhesion capacity”, Cytotherapy 2010, Vol. 11, pp. 251-259.
Rivière et al., “Magnetic targeting of nanometric magnetic fluid liposomes to specific brain intravascular areas”, Radiology 2007, Vol. 244, pp. 439-448.
Taboada et al., “Relaxometric and Magnetic Characterization of Ultrasmall Iron Oxide Nanoparticles with High Magnetization. Evaluation as Potential T1 Magnetic Resonance Imaging Contrast Agents for Molecular Imaging”, Langmuir 2007, Vol. 23, pp. 4583-4588.
Song et al., in “Using a Neodymium Magnet to Target Delivery of Ferumoxide-labeled Human Neural Stem Cells in a Rat Model of Focal Cerebral Ischemia”, Human Gene Therapy—2010, Vol. 21, pp.: 603-610.
Kyrtatos et al., “Magnetic Tagging Increases Delivery of Circulating Progenitors in Vascular Injury”, J. Am. Coll. Cardiol. Into. 2009, Vol. 2, pp.: 794-802.
Sun et al. “Monodisperse Mfe₂O₄ (M=Fe, Co, Mn) Nanoparticles” J. Am. Chem. Soc.—2004, Vol. 126, pp. 273-279.
Idalia Bilecka et al. “Simultaneous formation of ferrite nanocrystals and deposition of thin films via microwave-assisted nonaqueous sol-gel process”, J. Sol-Gel Sci. Technol—2010, Vol. 57, Number 3, pp. 313-322, [Online early access], DOI: 10.1007/s10971-010-2165-1.
US2011070202.
Thau-Zuchman et al. “Vascular endothelial growth factor increases neurogenesis after traumatic brain injury”, Journal of Cerebral Blood Flow & Metabolism—2010, Vol. 30, pp.: 1008-1016.
Valable et al, “VEGF-induced BBB permeability is associated with MMP-9 activity increase in cerebral ischemia: both effects decreased by Ang-1”, Journal of Cerebral Blood Flow & Metabolism—2005, Vol. 25, pp.: 1491-1504.
Zhang et al, “VEGF enhances angiogenesis and promotes blood-brain barrier leakage in the ischemic brain”, J. Clin. Invest—2000, Vol. 106, pp.: 829-838.

1. A method for treating a subject suffering a cerebrovascular accident comprising administering a composition comprising one or more endothelial progenitor cells (EPC) comprising superparamagnetic particles with a hydrodynamic diameter measured by dynamic light scattering comprised between 3 nm and 10 μm.

2. The endothelial progenitor cell (EPC) for use in the method according to claim 1, wherein the hydrodynamic diameter is an homogeneous hydrodynamic diameter measured by dynamic light scattering comprised between 3 nm and 10 μm said homogeneity determined as the deviation standard of the particle population size and which is lower than 60%.

3. The endothelial progenitor cell for use in the method according to claim 1, wherein the particles have a hydrodynamic diameter from 3 nm to 5 μm.

4. The endothelial progenitor cell for use in the method according to claim 1, wherein the particles have a diameter range from 15 nm to 60 nm.

5. The endothelial progenitor cell for use in the method according to claim 1, wherein the particles are disposed in an aggregate of sub-particles.

6. The endothelial progenitor cell for use in the method according to claim 5, wherein one or more of the particles comprise a core of magnetite and/or maghemite sub-particles, and an electrostatic coating comprising molecules with negative charge.

7. The endothelial progenitor cell for use in the method according to claim 6, wherein the molecules with negative charge are the same or different and are selected from the group consisting of citrate, and tetramethylammonium hydroxide.

8. A superparamagnetic nanoparticle obtainable by a process comprising the following steps:
a) mixing in an inert atmosphere an organic iron precursor selected from the group consisting of iron acetylacetonate and iron pentacarbonyl with 1,2-hexadecanediol and a surfactant system in an organic solvent and refluxing the mixture at a temperature comprised between 270° C. and 350° C. for a period of time from 0.5 to 2.0 hours to obtain a superparamagnetic iron oxide black precipitate;
b) dissolving the precipitate of step a) in an organic solvent in the presence of the surfactant system to obtain a dispersion of superparamagnetic iron oxide nanoparticles with a coating comprising the surfactant system;
c) adding while stirring an aqueous solution of the electrolyte tetramethylammonium hydroxide to the nanoparticle dispersion of step b);
d) adjusting the pH of the dispersion between 8 and 10 by adding dropwise with drop sizes from 25 to 200 μl and in a time span from 30 seconds to 3 minutes, a 0.01 M aqueous solution of an inorganic acid to form nanoparticles having a hydrodynamic diameter comprised between 10 nm and 100 nm measured by the Dynamic Light Scattering technique, said nanoparticles being disposed in a core of an aggregate of sub-particles of superparamagnetic iron oxide and a coating of the electrolyte tetramethylammonium hydroxide;
e) adding an aqueous solution of a citrate salt to a final concentration of citrate molecules per nm² of the nanoparticle surface, N, comprised between 0.5 to 40, said nanoparticle surface determined as 4·(r_{aggregate of sub-particles})², wherein r is the hydrodynamic radium of the aggregate of sub-particles; and
f) further adjusting the solution of step e) to a pH comprised between 6.5 and 7.5 by adding, dropwise with drop sizes from 25 to 200 μl, a 0.01 M aqueous solution of an inorganic acid.

9. The superparamagnetic nanoparticle according to claim 8, wherein the citrate salt is selected from the group consisting of sodium citrate and potassium citrate.

10. The superparamagnetic nanoparticle according to claim 8 with a hydrodynamic diameter comprised between 15 nm and 60 nm.

11. An endothelial progenitor cell comprising a superparamagnetic iron oxide nanoparticle, said nanoparticle comprising a core of an aggregate of sub-particles of superparamagnetic iron oxide and a coating comprising citrate, said superparamagnetic iron oxide nanoparticle having a hydrodynamic diameter comprised between 10 and 25 nm.

12. The endothelial progenitor cell of claim 11, wherein the superparamagnetic iron oxide nanoparticle has a hydrodynamic diameter comprised between 10 and 20 nm.

13. The endothelial progenitor cell according to claim 11, wherein the superparamagnetic iron oxide nanoparticle has a hydrodynamic diameter comprised between 10 and 15 nm.

14. The cell according to claim 11, comprising an iron concentration per cell comprised between 1 pg Fe/cell to 40 pg Fe/cell.

15. A pharmaceutical composition comprising one or more of the nanoparticles as defined in claim 8 and any pharmaceutically acceptable excipients.

16. The pharmaceutical composition of claim 15 which is an injectable composition.

17. A pharmaceutical composition comprising an EPC cell as defined in claim 11 and any pharmaceutically acceptable excipients.

18. The pharmaceutical composition of claim 17 which is an injectable composition.

19. A method for treatment of a cerebrovascular accident comprising administering to a subject in need thereof a composition comprising one or more endothelial progenitor cells as defined in claim 11.
